(19) United States
(12) Patent Application Publication
(10) Pub. No.: US 2025/0009545 A1
(43) Pub. Date: Jan. 9, 2025

(54) ORTHOPEDIC PRECAST WITH THERMAL COVER FOR CUSTOM OTHOSIS CREATION

(71) Applicant: Aspen Medical Products, LLC, Irvine, CA (US)

(72) Inventors: Joel Perez, Long Beach, CA (US); Albert V. Romo, Lakewood, CA (US)

(21) Appl. No.: 18/448,921

(22) Filed: Aug. 12, 2023

Related U.S. Application Data
(63) Continuation-in-part of application No. 17/673,716, filed on Feb. 16, 2022.
Application No. 17/673,716 is a continuation-in-part of application No. 17/178,071, filed on Feb. 17, 2021, now Pat. No. 11,964,061.

(51) Int. Cl. A61F 5/02; A61L 15/14

(52) U.S. Cl. CPC A61F 5/028; A61L 15/14

(57) ABSTRACT

One embodiment relates to an orthosis customization system featuring an orthopedic precast and a thermal cover. The orthopedic precast comprises a first section a plurality of interwoven strands of a first thermoplastic material having a first melting temperature. The thermal cover is configured to (i) preclude heat equal to or exceeding the first melting temperature onto at least a portion of the first section of the orthopedic precast including the plurality of interwoven strands of the first thermoplastic material to cause the portion of the first section to remain in a flexible state and a remainder of the first section of the orthopedic precast, after cooling, to transition from the flexible state to a rigid state as the orthopedic precast is converted into an orthosis.

### CROSS-REFERENCE TO RELATED APPLICATIONS

This application is a Continuation-in-Part of U.S. patent application Ser. No. 17/673,716 filed Feb. 16, 2022, which is a Continuation-in-Part of U.S. patent application Ser. No. 17/178,071 filed Feb. 17, 2021, the entire contents of both of these applications are incorporated by reference herein.

### FIELD

Embodiments of the disclosure relate to an orthopedic precast with customized thermal covers for placement over the orthopedic precast prior to generation of a custom orthosis triggered by an event.

### BACKGROUND

The following description includes information that may be useful in understanding the present invention. It is not an admission that any of the information provided herein is prior art or relevant to the presently claimed invention, or that any publication specifically or implicitly referenced is prior art.

Orthopedic braces (orthoses) usually need to be adjusted or customized in some manner to conform to the body part(s) being braced. A typical orthosis commonly has at least two portions, a rigid portion operating as a support structure and a flexible portion securing the orthosis to the body. The flexible portion often involve a strap, and in many orthoses, multiple straps are required to adequately secure the orthosis. It can be time-consuming for a patient to repeatedly have to adjust the different straps.

Custom orthoses have been made from a combination of resilient and flexible materials. For example, U.S. Pat. No. 9,572,703 to Matthews describes an orthosis sock that utilizes a resilient material for restricting movement of a patient's foot. While the orthosis is easier to wear than a typical ankle-foot-orthosis (AFO), the material forming the orthosis is not resilient and is insufficient to provide adequate support. The stitching of material to provide necessary resiliency is both labor intensive and susceptible to potentially uncomfortable areas at the stitching points.

Currently, custom orthoses are formed by creating a negative mold of a patient's body part, such as an ankle and foot for example, using the negative mold to create a positive mold, wrapping preheated flexible and hardenable materials about different portions of the positive mold, applying vacuum to the material-wrapped positive mold, and then allowing time for the materials to cool to the shape of the positive mold. Once cooled, the materials must be carefully cut off the mold then all cut edges must be ground/smoothed to the final shape. Production of such custom orthoses requires considerable skill, and is therefore relatively expensive.

An orthopedic precast for the generation of a custom orthosis, formed by interwoven sections of material that may conduct phase transitions from a flexible into a rigid composition through heat and shielding applications, is needed.

### BRIEF DESCRIPTION OF THE DRAWINGS

FIG. 1A is a perspective view of an exemplary embodiment of an orthosis customization system featuring an orthopedic precast and a thermal cover to concentrate phase transition of the material forming the orthopedic precast at one or more prescribed locations.

FIG. 1B is a perspective view of an exemplary embodiment of an orthosis generated from the orthopedic precast of FIG. 1A.

FIG. 2A is a perspective view of an exemplary embodiment of an orthopedic precast for generation of a leg-ankle-foot orthosis along with a thermal cover to impede phase transition of the material forming the orthopedic precast at one or more prescribed locations.

FIG. 2B is a perspective view of an exemplary embodiment of the leg-ankle-foot orthosis generated from the orthopedic precast of FIG. 2A.

FIG. 3A is an exemplary embodiment of dorsal and palmar views of an orthopedic precast for generation of a left wrist orthosis along with an attachable thermal cover to impede phase transition of material forming the orthopedic precast at one or more prescribed locations.

FIG. 3B is a perspective view of an exemplary embodiment of the left wrist orthosis generated from the orthopedic precast of FIG. 3A.

FIG. 4 is a perspective view of an exemplary embodiment a heating process of an embodiment of an orthopedic precast for conversion to an orthosis.

FIG. 5A is a schematic of an exemplary embodiment of an interwoven section of an orthopedic precast as illustrated in FIGS. 1A-3B.

FIG. 5B is a schematic of an exemplary embodiment of a cover being applied to a portion of the interwoven section of the orthopedic precast to prevent a phase transition caused by an application of heat from a thermal unit.

FIG. 6A is a schematic of an exemplary embodiment of interwoven sections of an orthopedic precast with a first interwoven section configured with a higher concentration of a thermoplastic material than in a second interwoven section with a cover to control phase transition in portions of the first interwoven section when heat applied.

FIG. 6B is a schematic of an exemplary embodiment of interwoven sections of an orthopedic precast having a first interwoven section configured with a tighter interweave than in a second interwoven section with a cover to control phase transition in one or more portions of the first interwoven section when heat applied.

FIG. 7A is an exemplary embodiment of an orthopedic precast for generation of a knee orthosis with areas of different thermoplastic materials along with an attachable thermal cover to impede phase transition of material forming the rigid knee protector.

FIG. 7B is a perspective view of an exemplary embodiment of the knee orthosis generated from the orthopedic precast of FIG. 7A.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include any combination of A, B, C, and/or D, even if not explicitly disclosed.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

### I. General Summary

Embodiments of the disclosure are directed to an orthopedic precast and methods in which an orthosis generated from the orthopedic precast may be structurally modified based, at least in part, on (i) a prescribed amount of heat (e.g., thermal energy such as heated air, heated steam, etc.) applied to the orthopedic precast and (ii) usage of a thermal cover designed to preclude heat from penetrating and/or focus heat to a prescribed area of the orthopedic precast. In particular, one or more sections of the orthopedic precast, in response to an application of heat at or exceeding a prescribed temperature followed by cooling, may experience phase transitions from a flexible material into a rigid material. The phase transitions are performed to form an orthosis with rigid sections such as an ankle-foot-orthosis (AFO), cervical orthosis, torso orthosis, leg-ankle-foot orthosis, wrist guard, or shoulder orthosis for example.

Herein, the application of heat to one or more sections of the orthopedic precast may cause some or all of the materials within the section(s) to undergo a phase transition (change), namely the melting, reforming, and/or bonding with other materials before solidifying after cooling to create a rigid structure for those section(s). In order to deflect or redirect heat away certain sections of the orthopedic precast and toward other sections, thermal covers may be produced with patterns to allow heat to be directed to targeted sections inclusive of strands of thermoplastic material to alter the rigidity of these sections from a flexible state to a rigid state, when the rigid section is part of an orthosis formed from the orthopedic precast. The thermal cover may be attached to the orthopedic precast through any type of fastening scheme including adhesive, unbroken loop (UBL) material, clips, brackets, snaps, or any other fasteners.

According to one embodiment of the disclosure, the orthopedic precast may feature (i) a first section comprising one or more interwoven strands inclusive of at least a first thermoplastic material that is coupled to (ii) a second section comprising one or more interwoven strands inclusive of the same or different thermoplastic material and/or non-thermoplastic material. For example, the orthopedic precast may include regions of differing materials such as thermoform material to result in rigid portion(s) along a back (posterior) and/or bottom area of the orthosis and nylon/elastic materials placed in regions to result in flexible portion(s) along a front (anterior) or top area of the resultant orthosis. Same heat delivered everywhere for ease of method, but the front/top nylon/elastic material does not undergo a phase transition into a rigid form. The posterior and/or back areas may became rigid.

Herein, thermal covers may be pre-fabricated and designed to shield heat directed to the second section (or a portion of the second section) while allowing heat to be applied to the first section of the orthopedic precast. Alternatively, the thermal covers may be produced with a desired structure, but these covers may be modified (e.g., cut or bent in shape to change coverage) in order to further (and more precisely) transform the orthopedic precast into a custom orthosis.

As an illustrative example, the first interwoven section and the second interwoven section may be coupled together as part of a single layer construction, where these interwoven sections may utilize different materials (e.g., different thermoplastic materials, thermoplastic and non-thermoplastic materials, etc.) and/or different interweaving techniques to provide greater rigidity or maintain a flexible and/or elastic construction after heating the interwoven sections to a temperature at or beyond a melting temperature for certain thermoplastic materials. According to one embodiment of the disclosure, a thermal cover may be used to (i) concentrate thermal energy toward a portion of the first interwoven section to melt strands of the first thermoplastic material so as to increase rigidity at the portion of the first interwoven section after cooling and/or (ii) reduce (or prevent) thermal energy toward neighboring portions so that these portions remain more flexible in composition.

Alternatively, the first interwoven section and/or the second interwoven section may be layered on each other to provide additional rigidity at certain locations of the orthosis proximate to particular body parts of the patient. The thermal cover may be used reduce (or prevent) thermal energy toward certain portions of the first interwoven section so that these portions remain more flexible in composition than the other regions exposed to the thermal energy.

As an illustrative example, a thermal unit (e.g., heating source) may direct thermal energy (e.g., heated steam, heated air, etc.) to the orthopedic precast when creating a custom orthosis. For this example, the orthopedic precast includes (i) a first section interwoven, at least in part, with strands of a first thermoplastic material and (ii) a second section interwoven, at least in part, with strands of the first thermoplastic material. A thermal cover may be positioned between the thermal unit and the second interwoven section of the orthopedic precast.

When the thermal source provides thermal energy (heat) that reaches or exceeds a melting temperature of the first thermoplastic material, the first interwoven section experiences a phase transition. This phase transition may involve a partial melting one or more portions of the first thermoplastic material. After experiencing a phase transition, the first interwoven section (or a portion thereof) transitions from a flexible state to a rigid state (referred to as a “shell section” for the resultant orthosis). Despite the thermal unit providing thermal energy that reaches or exceeds the melting temperature of the first thermoplastic material, the second interwoven section does not experience a phase transition, but retains its flexible (fabric-like) characteristics.

Herein, besides through the thermal cover, different interwoven sections and different regions within the same interwoven section may experience different amounts of phase transition (if any), depending on their material composition, structure, and the amount of heat transferred to the interwoven section. The different amounts of phase transition allow for different portions of the resultant orthosis to be configured with different levels of rigidity (e.g., rigid, flexible, etc.). For instance, where a second portion of the first interwoven section includes the same thermoplastic material as a first portion of the first interwoven section, but in lesser quantity (e.g., lesser strand numbers, thinner thermoplastic strands, lesser volume of thermoplastic strands due to interweaving/stitching pattern, etc.), after heating to cause a phase transition for thermoplastic material, the first portion may become more rigid than the second portion. Similarly, for the situation where the second portion includes a second thermoplastic material that is different from and has a higher melting temperature than the first thermoplastic material included as part of the first portion, the second interwoven section may remain flexible when heat (below the melting temperature of the second thermoplastic material and higher than the melting temperature of the first thermoplastic material) is applied to the first interwoven section.

As described above, a thermoplastic material used in one interwoven section can be different from or the same as a thermoplastic material used in another interwoven (shell) section. According to one embodiment of the disclosure, the temperature needed to activate the phase transition of the thermoplastic material within an interwoven section may range from 50° Celsius (C) to 140° C. The difference in activation temperatures (e.g., melting temperatures of different thermoplastic material within the same or different interwoven sections may differ by 10°-20° C., 10°-30° C., 30°-50° C., or the like.

In some embodiments, the thermoplastic material composition can comprise at least 30 wt % of the orthopedic precast. For example, the thermoplastic section can comprise between 5 wt % and 90 wt % of the orthopedic precast, such as between 50 wt % and 90 wt % of the orthopedic precast.

In some embodiments, the rigid or flexible sections can be layered. For example, the flexible section can be positioned adjacent to (e.g., laminated with) at least a layer of the rigid section that provides structural reinforcement for the orthosis. Alternatively, a rigid section can be positioned adjacent to (e.g., laminated with) at least one layer of a flexible section to enhance skin comfort.

### II. Terminology

As used herein, the terms “interweaved,” “interweaving,” “interwoven” or other tenses, when used in connection with material, are generally defined as any arrangement of material substantially comprising one or more strands of material (e.g., thread, yarn, etc.) to produce a section of an orthopedic precast. The arrangement of material may include one or more types of materials attached together as a single layer of continuous material or as multiple (two or more) layers of material. This attachment may occur through an interweaving process that includes knitting, stitching (e.g., “V” shaped stiches), weaving (interlacing strands of material), crocheting (knot-like stitches), macramé (knot-like stiches in geometrical patterns), and/or even topical application such as spray-on application of thermoplastic material to alter the composition of the material such as altering a non-thermoplastic material to a thermoplastic material.

Furthermore, this interweaving of material may be performed in accordance with a two-dimensional (2D) interweaving process or a three-dimensional (3D) interweaving process. The 2D interweaving process allows for the creation of a low-profile orthoses where the shell section and flexible section are interwoven to form a single layer of material. The 3D interweaving process may be utilized to create a layered interwoven orthosis, such as added padding and/or additional layers of the same or different types of thermoplastic material may be added to the shell section to increase its rigidity. Also, the increased layering provides increased density for larger-sized orthoses or in certain areas of the orthoses that need increased density (e.g., hip area, etc.).

As used herein, the terms “rigid” or “rigidity” with respect to an orthopedic precast or a section of the orthopedic precast means that the rigid object or rigid section of the object will resist bending or deformation. According to this definition, different lengths of a given structure and composition can be rigid at a shorter length, and flexible at a longer length. As an illustrative example, in some cases, a “rigid” object or “rigid” section(s) of the object may represent that the object or section of the object may be deformed, in some cases permanently, if bent or twisted by a predetermined angle (e.g., at least 30° end to end). Therefore, a “rigid” object may have levels of rigidity, where the rigid object could also be referred to as a “semi-rigid” object or “semi-rigid” section(s) of the object where bending may not cause permanent deformation. As used herein, the terms “bent” or “bending” may be construed to applying an angular deformation, which may include twisting.

As used herein, the term “flexible” with respect to an orthopedic precast or a section of the orthopedic precast may signify that the orthopedic precast or the orthopedic precast section will not be permanently deformed by bending. For example, an interwoven section of an orthopedic precast may be flexible in nature, even after a heating and cooling process. As used herein, the term “permanently deformed” means that deformation remains unless the deformation is actively repaired. According to this definition, the orthopedic precast or a section of the orthopedic precast could be rigid in one direction, and flexible in another direction. Unless otherwise specified in such cases, the orthopedic precast or section of the orthopedic precast is deemed to be rigid.

As used herein, the term “elastic” with respect to an orthopedic precast or section of the orthopedic precast means that if the elastic section is stretched or compressed lengthwise by at least 10%, it will return to its resting length, without the need for application of an external force, and without permanent deformation.

As used herein, the term “shell” means a structure configured to impart rigidity that restrains movement of a part of a patient's body, wherein the structure is either (i) rigid after an application of a prescribed heating and cooling phase or (ii) will become rigid upon completion of that heating and cooling phase. In particular, an interwoven section of an orthopedic precast existing in a non-rigid state may constitute a shell section by having characteristics that allow this interwoven section to transition from its non-rigid form to a rigid form in response to an application of heat (heated air, steam, etc.) that is equal to or exceeds a prescribed melting temperature and subsequent cooling. In some embodiments, the shell may include a structure having a cavity, hollow, or lumen.

As used herein the term “patient” includes both humans and animals, independently of whether the patient is under the care of a medical or veterinary professional.

As used herein, the term “strand” is generally defined as an elongated length of one or more natural, artificial, or combined natural and artificial substances. According to one embodiment of the disclosure, each strand may be a fiber no more than 3 millimeters (mm) thick over a length of at least one centimeter (cm), although other sizing is contemplated. Examples of a “strand” may include thread, yarn, string, cord, or any flexible material that may be organized into a structure (e.g., stitched, weaved, etc.) to produce an object such as an orthopedic precast. Where the strand or strands denote thermoplastic material, these strands may be elongated fibers of the thermoplastic material, or alternatively, the strand may be another substance at least partially coated with thermoplastic material or impregnated with thermoplastic material.

In some embodiments, the orthopedic precast may be transformed into an orthosis having a tubular construction that includes both shell and flexible sections. In some embodiments, the orthosis may be configured with a tubular construction that includes shell sections of different levels of rigidity.

In some embodiments, the shell section orients lengthwise along the tube. For example, the shell and flexible sections of an orthopedic precast could correspond to the anterior and posterior sections of a lower leg respectively, and these sections can be directly connected to each other. For such an orthopedic precast, the shell section would be considered to be oriented lengthwise along the tube. Alternatively, a shell section can orient crosswise with respect to a tube. For example, an orthopedic precast capable of accommodating a torso section of a patient could have a shell section that extends across the front of a patient, and flexible sections that also extend across the front of the patient, connected superiorly and inferiorly to the shell section. For such an orthosis, the shell section would be considered to be oriented crosswise along the tube.

As used herein, the term “crosswise” includes various degrees of diagonality.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects, and advantages of the inventive subject matter will become more apparent from the following detailed description of preferred embodiments, along with the accompanying drawing figures in which like numerals represent like components.

### I. Illustrative Orthopedic Precasts/Resultant Orthoses

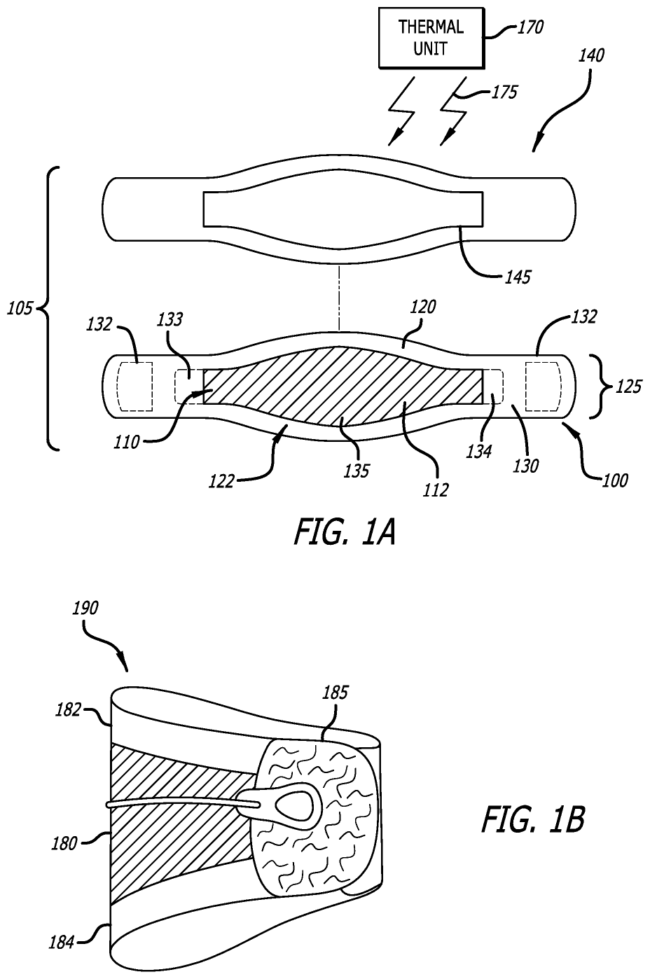

Referring to FIG. 1A, a perspective view of an exemplary embodiment of an orthosis customization system 105 is shown, where the orthosis customization system includes an orthopedic precast 100 and a thermal cover 140 to concentrate phase transition of material forming the orthopedic precast 100 at one or more prescribed locations. Herein, operating as a lumbar sacral orthosis (LSO) or part of a thoracic lumbar sacral orthosis (TLSO), the orthopedic precast 100 may be configured with a first section 110 that is formed with strands of thermoplastic material 112, which transitions from a flexible state to rigid state (referred to as a “shell section 180” of FIG. 1B) after a prescribed amount of heat is applied thereto and subsequently cooled. This rigidity may be associated with movement in a first direction (e.g., vertically) while lateral flexibility is retained so that a resultant orthosis 190 can be laterally adjusted to surround hip areas of the patient.

Positioned along an upper and lower edges of the first interwoven section 110, upper and lower interwoven sections 120 and 122 may feature one or more strands comprising non-thermoplastic material that retain their flexible construction even after heating (each referred to as a “flexible section”). Collectively, as shown, the interwoven sections 110, 120 and 122 generally compose a tube (e.g., belt) 125, with two arm members. In this particular example, both upper and lower sections 120 and 122 are oriented on opposite outer edges of the first interwoven section 110 to create a transitional area therefrom. This transitional area is intended, when the orthopedic precast 100 is formed into the orthosis 190 of FIG. 1B, to provide a more comfortable fit of the orthosis 190.

According to one embodiment of the disclosure, the strands of the thermoplastic material 112 forming the first interwoven section 110 may include a single type of thermoplastic material. Alternatively, the strands 112 forming the first interwoven section 110 may constitute a composite of materials, which may include at least two different thermoplastic materials, where the thermoplastic materials may have the same or different melting temperatures. As yet another alternative embodiment of the disclosure, the strands 112 forming the first interwoven section 110 can also be an interwoven composite of one or more thermoplastic strands and one or more non-thermoplastic materials, provided the rigidity of the resultant shell section 180 of FIG. 1B is altered after a selected temperature and duration of heat is applied and the first interwoven section 110 is cooled.

The thermal cover 140 may be constructed in accordance with regions of the orthopedic precast 100 that are targeted to receive thermal energy 175 and targeted to be shielded from the thermal energy 175. More specifically, as shown, the thermal cover 140 includes an opening 145 sized so when, a thermal unit 170 is applying the thermal energy 175 (e.g., heated steam, heated air, etc.) to a top surface 130 of the orthopedic precast 100, the thermal cover 140 enables the thermal energy 175 to be applied to most of the strands of the thermoplastic material 112 within the first interwoven section 110 (e.g., strands within portion 135). However, the thermal cover 140 precludes the transfer of thermal energy 175 to portions of the first interwoven section 110 such as portions 132-134. As a result, portions 132-134 of the first interwoven section 110 retain their flexible (fabric-like) characteristics while the portion 135 is configured to become more rigid.

Referring still to FIG. 1A, the strands of thermoplastic material 112 may be formed of thermoplastic material in their entirety or these strands 112 may be formed of a different material coated and/or impregnated (through an application process) with one or more thermoplastic materials. Herein, a “strand of thermoplastic material” may reference a strand formed of material inclusive of thermoplastic material that, when heated and cooled, alter the overall rigidity of the material while a “strand of non-thermoplastic material” may reference a strand of material devoid of thermoplastic material that affects its rigidity.

According to one embodiment of the disclosure, the thermoplastic materials may be configured to form flexible strands at room temperature, are non-toxic, melt between a prescribed thermal range (e.g., thermal range between 140° C. and 350° C.), and become rigid when strands are partially melted together into a sheet or mat having a thickness of 0.5 mm to 6 mm. Contemplated examples of thermoplastic materials may include, but are not limited or restricted to the following: Polyethylene Terephthalate (PET), Polyether ether ketone (PEEK), Polyphenylene oxide (PPO), Polypropylene (PP), polyethylene (PE), polyvinyl chloride (PVC) and polystyrene (PS), poly(methyl methacrylate) (PMMA), Acrylonitrile butadiene styrene (ABS), Polylactic acid (PLA), Polybenzimidazole (PBI), Polycarbonate (PC), Polyether sulfone (PES), Polyoxymethylene (POM), Polyphenylene sulfide (PPS), Polystyrene, Polyvinyl chloride (PVC), Polyvinylidene fluoride (PVDF), Polytetrafluoroethylene (PTFE), Polyamide 6 (PA6), Polybutylene terephthalate (PBT), Polyetherimide (PEI), or the like.

The thermoplastic and non-thermoplastic materials can be selected using any combination of natural and synthetic materials to accomplish a desired characteristic, as for example, a desired degree of stiffness, compressibility, flexibility, bending, stretch, and resilience. As described above, one or more strands associated with the first interwoven section 110 may include both thermoplastic and non-thermoplastic materials. For example, as an illustrative embodiment, the non-thermoplastic material may include Kevlar™ to increase the durability/toughness of the shell section 180 of the resultant orthosis 190, as one or more other non-thermoplastic materials (e.g., cotton fibers, non-carbon fibers, nanotubes, glass fibers, ceramic, and/or metal fibers) may be used.

Similarly, as another illustrative embodiment, one or more strands of material forming the upper/lower interwoven section(s) 120 and/or 122 can also comprise non-thermoplastic materials to retain elasticity. However, according to this embodiment of the disclosure, the upper/lower interwoven section(s) 120 and/or 122 may include strands of thermoplastic material equivalent to the strands of thermoplastic material 112 within the first interwoven section 110, provided (i) the thermal cover 140 is positioned to cover regions of the orthopedic precast 100 including of the upper/lower interwoven sections 120 and 122 and/or (ii) the strands of thermoplastic material forming the upper/lower interwoven sections 120 and 122 have a higher melting temperature that is substantially above the melting temperature of the strands of thermoplastic material 112 interwoven in the first interwoven section 110.

In production, the orthopedic precast 100 may be placed over a positive mold when heated, such that at least some of the thermoplastic material(s) fuse, in what will become a rigid shell section 180 for the orthosis 190 as shown in FIG. 1B. This allows the shell section 180 to closely conform to whatever part(s) of the patient are to be motion-restrained. Alternatively, the orthopedic precast 100 may be placed over a first positive mold, removed inside-out to provide a different (opposite) layering scheme for this flipped precast, which is placed over a second positive mold, heated, and subsequently cooled to form the orthosis 190 with the rigid shell section 180 having multiple layers.

According to this embodiment of the disclosure, the orthopedic precast 100 has a tubular configuration, with superior and inferior open ends. However, as depicted in FIG. 1B, the orthosis 190 can also open and close laterally, using a fastener such as a Velcro™ or similar hook and loop fastener 185, which can be installed at the orthopedic precast stage. Any suitable fasteners are contemplated, including buttons, toggles, studs, snap fastener, poppers, buckles, zippers, frogging, hooks and eyes, magnets, grommets, brooches, safety pins, fabric ties, and laces.

As shown in FIGS. 1A-1B, the upper/lower interwoven sections 120 and 122 can advantageously be elastic, and where these interwoven sections 120 and/or 122 can be constructed with a lesser amount of thermoplastic material, thermoplastic material with higher melting threshold, or no thermoplastic material to be increasingly flexible and/or elastic towards the outer edges. Such variance in flexibility and elasticity can promote the patient's comfort by transitioning pressure against the body. Thereafter, the flexible sections 182 and 184 of the orthosis 190 may remain elastic even after the heating and cooling processes. Elasticity is advantageous because it causes the orthosis 190 to conform to different body shapes. Moreover, since the shell section 180 of the orthosis 190 has limited extendibility, the flexibility of the shell section 180 could be sufficient to allow a user to conform the orthosis 190 around her or his waist.

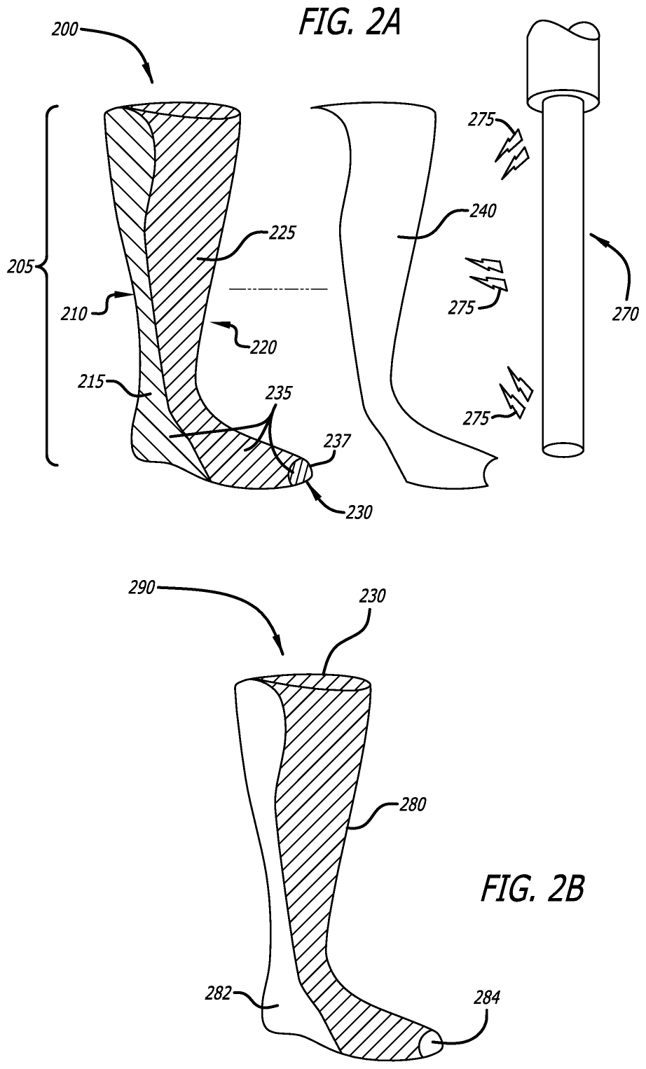

Referring now to FIG. 2A, a perspective view of an exemplary embodiment of an orthosis customization system 205 including an orthopedic precast 200 and a thermal cover 240 for generation of a leg-ankle-foot orthosis 290 (see FIG. 2B). The orthopedic precast 200 includes a plurality of sections such as a first section 210, a second section 220, and a third section 230. The first section 210 includes a collection of interwoven strands of a first thermoplastic material 215 situated proximate to an ankle-heal area and proximate to a calf area of a patient for this example. The thermal cover 240 is configured to impede phase transition of a second thermoplastic material 225 interwoven to form the second section 220 of the orthopedic precast 200, except at the third interwoven section 230 situated near a toe region of the orthopedic precast 200.

More specifically, as shown in FIG. 2A, the thermal cover 240 may be constructed in accordance with regions of the orthopedic precast 200 that are targeted to receive thermal energy 275 from a thermal unit 270 and targeted to be shielded from the thermal energy 275. For instance, as shown, the thermal cover 240 is formed as a brace member attached to a leg of the patient or a positive mold to cover the second interwoven section 220. However, the first and third interwoven sections 210 and 230 remain uncovered. As the thermal unit 270 applies the thermal energy 275 (e.g., heat) to a top surface 235 of the orthopedic precast 200, the thermal cover 240 enables the thermal energy 275 to be applied to the strands of the first thermoplastic material 215 along with a portion 237 of the interwoven strands of thermoplastic material 225 near the toe area.

As a result, the second interwoven section 220 retains its flexible (fabric-like) characteristics to form a flexible portion 280 of the orthosis 290. The first interwoven section 210 along with the third interwoven section 230 undergoes a phase transition from their flexible (fabric-like) characteristics to rigid characteristics forming shell portions 282 and 284, respectively.

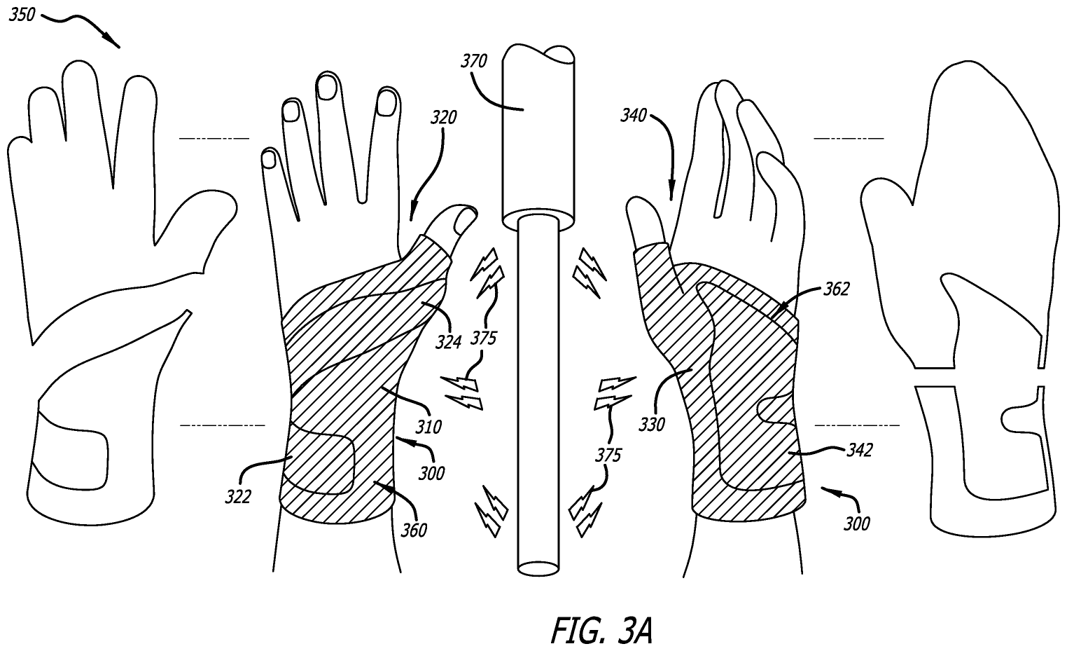

Referring now to FIG. 3A, an exemplary embodiment of dorsal and palmar views of an orthopedic precast 300 for generation of a left wrist orthosis 390 along with an attachable thermal cover 350 to impede phase transition of material forming the orthopedic precast 300 at one or more prescribed locations is shown. One or more thermal covers 350 and 355 may be attached to the orthopedic precast 300 by a fastening mechanism including, but not limited to a clip, strap, adhesive, fastener, or the like.

Herein, according to this embodiment of the disclosure, the orthopedic precast 300 features interwoven strands of thermoplastic material 310 situated on a dorsal side 320 of the orthopedic precast 300 and interwoven strands of thermoplastic material 330 situated on a palmar side 340 of the orthopedic precast 300. The strands of thermoplastic materials 310 and 330 may be the same thermoplastic material or different thermoplastic material with generally the same or different melting temperatures. A first thermal cover 350 is configured to attach itself to provide access to multiple physically separated regions 322 and 324 on the dorsal side 320 of the orthopedic precast 300 and a single region 342 on the palmar side 340 of the orthopedic precast 300.

As a thermal unit 370 applies thermal energy 375 (e.g., heat) to a top surface 360 of the dorsal side 320 of the orthopedic precast 300, the thermal cover 350 enables the thermal energy 375 to be applied to the interwoven strands of the thermoplastic material 310, notably those strands of thermoplastic material residing within the regions 322 and 324 on the dorsal side 320 of the orthopedic precast 300. As a result, as shown in FIG. 3B, rigid (shell) portions 380 and 382 are formed for the left wrist orthosis 390.

Similarly, as the thermal unit 370 applies thermal energy 375 (e.g., heat) to a top surface 362 of the palmar side 340 of the orthopedic precast 300, the thermal cover 350 enables the thermal energy 375 to be applied to the interwoven strands of the thermoplastic material 330, notably those strands of thermoplastic material residing within the region 342 on the palmar side 340 of the orthopedic precast 300. As a result, as shown in FIG. 3B, rigid (shell) portion 386 is formed, which are coupled to the rigid portions 380 and 382 on the dorsal side 320.

As a result, flexible portion 388 extends entirely around the shell portions 380, 382, and 386. On the orthosis for each hand, there are three openings, one for the thumb, one for the wrist, and one for the fingers/distal metacarpal region. In this particular example, a thumb spica 392 includes the shell portion 384 and the flexible portion 388. Herein, the resultant wrist orthosis 390 of FIG. 3B, corresponding in structure to the orthopedic precast 300, effectively reduces wrist flexion, extension, abduction, adduction, and rotational movement, while still being relatively easy to put on because of the flexible portion 388. The rigidity of the shell portions 380, 382, and 386 may be the same, or the rigidity between these shell portions 380, 383, and 386 may differ based on the type of thermoplastic material used, number and/or concentration of strands of the thermoplastic material within these shell portions, the interweaving stitch or pattern utilized, etc.

One advantage associated with this type of wrist orthosis 390 is that the patient could slide the orthosis 390 on as a glove with no tightening strap(s) required. With integrated regions of rigidity, the wrist orthosis 390 is configured to stabilize the wrist joint and/or the thumb without additional latching of straps or other fasteners. Heating of the orthopedic precast 300 may occur while positioned on the patient or by a mold.

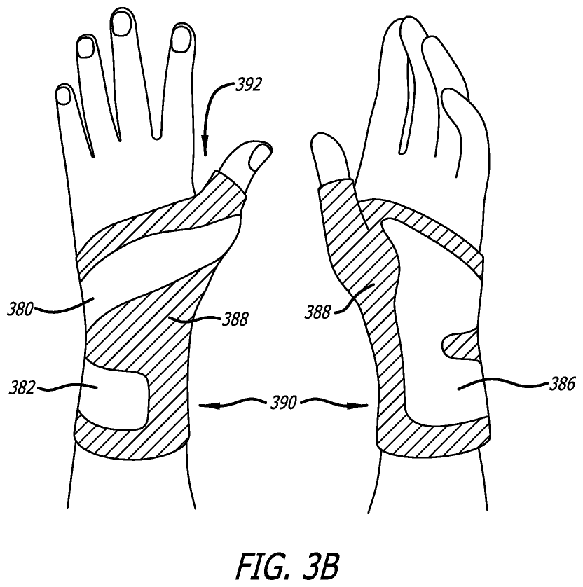

Referring now to FIG. 4, a perspective view of an exemplary embodiment a heating process of an embodiment of an orthopedic precast 400 for conversion to an orthosis 490 is shown. Herein, the orthopedic precast 400 is directed to a foot orthosis designed to protect a front foot area of a patient, albeit the development of the pre-fabricated orthosis may be directed to any part of a patient's body. Herein, as shown, the orthopedic precast 400 is formed with interwoven strands of thermoplastic material 410. Alternatively, the orthopedic precast 400 may be formed with interwoven strands of two different material such as different thermoplastic materials or a thermoplastic material and non-thermoplastic material. This configuration may require a different configuration for a thermal cover 440 used to control phase transitions occurring within the orthopedic precast 400.

Herein, according to this embodiment of the disclosure, the orthopedic precast 400 may be placed on a positive mold 420 of a targeted body part (or body part of a wearer). Alternatively, the orthopedic precast 400 may be placed on a patient (wearer) in lieu of the positive mold, provided a temperature for obtaining a desired rigidity within desired portions of the orthopedic precast 400 would fall within a temperature range that would not injure the patient. The thermal cover 440 may be placed over a portion of the targeted body part to which rigidity of the orthosis is unnecessary. For example, for foot protection after bunion surgery for example, a rigid covering 470 from the metatarsals to the distal phalange (e.g., a front portion of the foot) is needed. The remainder of the orthosis 490 can be made of flexible material. Therefore, the thermal cover 440 may be configured to position itself over a heal-ankle region 450, leaving a toe region 455 exposed.

Thereafter, the orthopedic precast 400 along with the thermal cover 440 may be heated to a temperature 460. The temperature 460 may be greater than or equal to a melting temperature of the interwoven strands of thermoplastic material 410 to allow for partial melting and hardening upon cooling. Once cooled, the orthopedic precast 400 becomes the custom orthosis 490, with the rigid covering 470 located in the region 455 exposed by the thermal cover 440 and a flexible portion 480 located in the region 450. This provides sufficient stiffness so that the orthosis 490 can retain its shape when removed from the positive mold (or wearer).

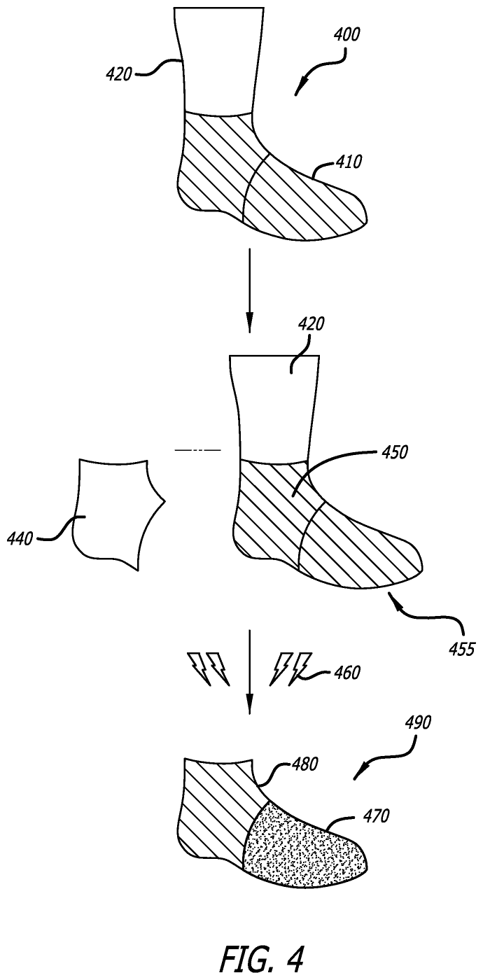

Referring to FIG. 5A, a schematic of an exemplary embodiment of an interwoven segment 510 of an orthopedic precast 500, such as precasts as illustrated in FIGS. 1A-3A, is shown. The orthopedic precast 500 features multiple layers of interwoven material, where the interweaving of the materials may be accomplished through a number of techniques, including complex multi-layering interweaving processes, separate interwoven sections positioned on each other and attached together, or a single layer of interwoven material that is folded over itself to form a multi-layered interwoven section.

As shown, a segment 510 of the orthopedic precast 500 including a first interwoven section 520 and a second interwoven section 530. The second interwoven section 530 features one or more strands of a second material 535, which may have a lower melting temperature than one or more strands of a first material 525 of the first interwoven section 520. For this illustrative embodiment, the different materials 525 and 535 may be a layered composition forming the orthopedic precast 500. For example, the strands of the second material 535 may be partially melted when a temperature above its melting temperature is applied, where this phase transition increases the rigidity of the thermoplastic material after cooling. Concurrent to the phase transition occurring for the strands of the second material 535, the strands of the first material 525 may experience no phase transition (e.g., no change from flexible to rigid state), where the first interwoven section 520 may provide comfort if placed against a skin of the patient.

As with all examples herein, the second material 535 of the second interwoven section 530 may constitute a thermoplastic material having a lower melting temperature than the first material 525 pertaining to the first interwoven section 520.

Referring now to FIG. 5B, a schematic of the segment 540 of an orthosis 590 generating from the interwoven segment 510 of the orthopedic precast 500 shown in FIG. 5A, following heating at or above the melting temperature of the thermoplastic material 535 of the second interwoven section 530, is shown. A thermal cover 550 is positioned to cover a portion of the interwoven segment 510, namely thermoplastic strands 560 being a subset of the strands of thermoplastic material 535. The thermal cover 550 precludes an application of heat to the thermoplastic strands 560 from a thermal unit 570, and thus, the thermoplastic strands 560 do not undergo a phase transition as occurred for a remainder (remaining strands 565) of the strands of the thermoplastic material 535. After cooling, the thermoplastic strands 560 retain some flexibility as the remaining strands 565 solidify to generate a rigid (shell) section within the segment 540 of the resultant orthosis 590.

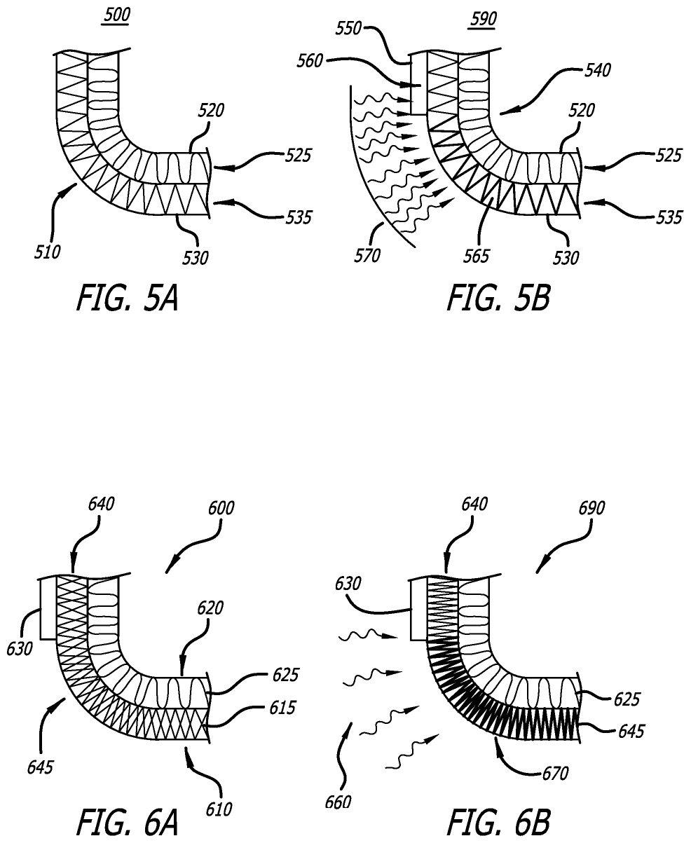

Referring to FIG. 6A, a schematic of an exemplary embodiment of a portion of an orthopedic precast 600, featuring a first interwoven section 610 and a second interwoven section 620, is shown. The first interwoven section 610 is configured with a higher concentration of interwoven strands of thermoplastic material 615 than strands of the thermoplastic material 625 included in the second interwoven section 620. Herein, a thermal cover 630 may be situated to prevent a first subset 640 of the interwoven strands of thermoplastic material 615 (hereinafter, “first thermoplastic strand series 640”) from undergoing a phase transition when heat is applied. A second subset 645 of the interwoven strands of thermoplastic material 615 (hereinafter, “second thermoplastic strand series 645”) is situated to undergo a phase change to transition from an original flexible (fabric-like) state to a rigid state.

According to this embodiment of the disclosure, as shown in FIG. 6B, upon applying heat 660 at or above the lower range of the melting temperature of the strands of thermoplastic material 615, the first interwoven section 610 may experience a partial phase change caused by partially melting of the second thermoplastic strand series 645 while the first thermoplastic strand series 640 remains in a flexible construction.

After cooling, an orthosis 690 has been produced from the orthopedic precast 600. For the orthosis 690, a region 670 associated with the second thermoplastic strand series 645 may solidify, becoming more rigid than the first thermoplastic strand series 640 as well as the strands of the thermoplastic material 625 included in the second interwoven section 620 of FIG. 6A, due to the first interwoven section 610 experiencing a greater volume of melted thermoplastic material due to its higher concentration level.

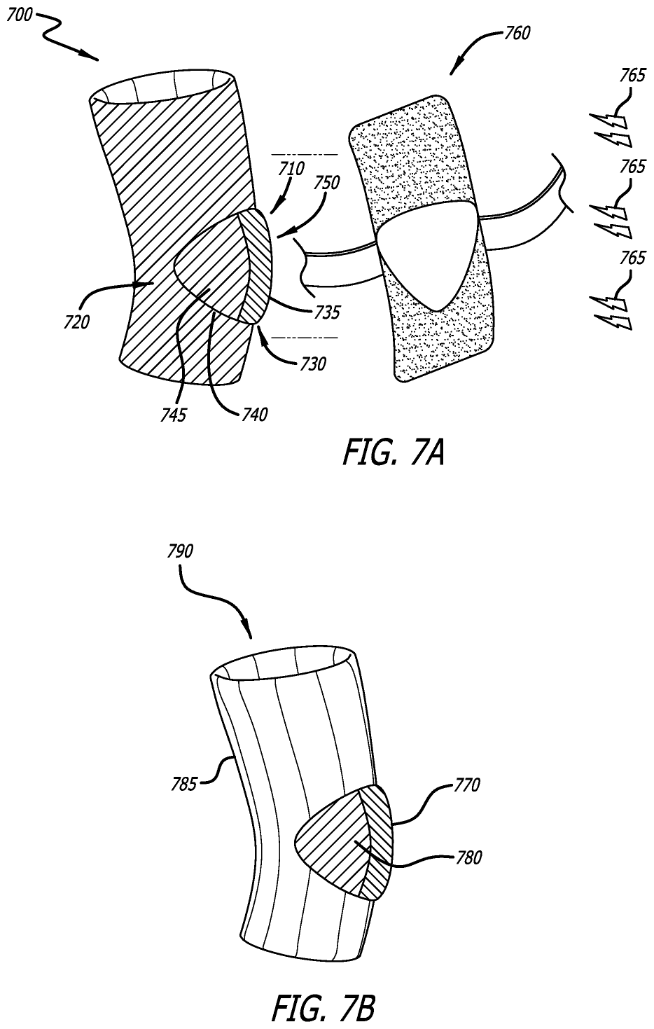

Referring now to FIG. 7A, a perspective view of an orthopedic sleeve precast 700 with a first sleeve section 710 and a second sleeve section 720. The first sleeve section 710 features a first subsection 730 including a plurality of interwoven strands of a first thermoplastic material 735 and a second subsection 740 including a plurality of interwoven strands of a second thermoplastic material 745. For this embodiment, the first thermoplastic material 735 possesses a lower melting temperature (T1) than a melting temperature (T2) for the second thermoplastic material 745. Herein, the first subsection 730 may be positioned with the second subsection 740 around a patella region 750, where the plurality of interwoven strands of the first thermoplastic material 735 are attached to the plurality of interwoven strands of the second thermoplastic material 745. The second interwoven section 720 may be formed with the second thermoplastic material 745 similar to the second subsection 740.

As shown in FIG. 7B, after thermal cover 760 is positioned to expose the first sleeve section 710 and cover a front-facing portion of the second sleeve section 720 and a prescribed amount of heat 765 (t, where T2≈t≥T1)) is applied to at least the first sleeve section 710, and after cooling, the first subsection 730 transitions to a rigid shell portion 770 formed to protect the patella from blunt forces. For this example, the first subsection 730 (inclusive of the plurality of interwoven strands of the first thermoplastic material 735 within the orthopedic sleeve precast 700) undergoes a phase change that results in formation of the shell portion 770 that is more rigid than a shell portion 780 produced by the second subsection 740 (inclusive of the plurality of interwoven strands of the second thermoplastic material 745 within the sleeve precast 700). The second interwoven section 720 within the orthopedic precast 700 remains flexible to constitute a flexible portion 785 of a resultant orthosis 790.

As a result, the orthopedic sleeve precast 700 may be transformed into a custom orthosis and knee protector featuring a protective shell portion integrated as part of the single interwoven composite layer.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. An orthosis customization system, comprising:
an orthopedic precast that comprises a first section including a plurality of interwoven strands of a first thermoplastic material having a first melting temperature; and
a thermal cover configured to (i) preclude heat equal to or exceeding the first melting temperature onto at least a portion of the first section of the orthopedic precast including the plurality of interwoven strands of the first thermoplastic material to cause the portion of the first section to remain in a flexible state and a remainder of the first section of the orthopedic precast, after cooling, to transition from the flexible state to a rigid state as the orthopedic precast is converted into an orthosis.

2. The orthosis customization system of claim 1, wherein the thermal cover is made of and configured to be coupled to the orthopedic precast prior to applying the heat to the orthopedic precast.

3. The orthosis customization system of claim 2, wherein the thermal cover includes an adhesive backing for coupling to a top surface of the orthopedic precast.

4. The orthosis customization system of claim 1, wherein the orthopedic precast further comprises a second section including a plurality of interwoven strands of a second thermoplastic material having a second melting temperature higher than the first melting temperature so that the second section remains in a flexible state when the orthopedic precast is converted into the orthosis.

5. The orthosis customization system of claim 1, wherein the orthopedic precast further comprises a second section including a plurality of interwoven strands of the first thermoplastic material having a tighter interweaving pattern than the plurality of interwoven strands of the first thermoplastic material associated with the first section.

6. The orthosis customization system of claim 5, wherein the second section transitions to a rigid portion of the orthosis having a greater rigidity than the remainder of the first section in response to an application of the heat being equal to or exceeding the first melting temperature onto both the first section and the second section.

7. The orthosis customization system of claim 1, wherein the orthopedic precast further comprises a second section including a plurality of interwoven strands of the first thermoplastic material having a thickness in diameter greater than a thickness of the plurality of interwoven strands of the first thermoplastic material associated with the first section.

8. The orthosis customization system of claim 7, wherein the second section transitions to a rigid portion of the orthosis having a greater rigidity than the remainder of the first section in response to an application of the heat being equal to or exceeding the first melting temperature onto both the first section and the second section.

9. The orthosis customization system of claim 1 being a lumbar sacral orthosis (LSO) or part of a thoracic lumbar sacral orthosis (TLSO).

10. An orthosis customization system, comprising:
an orthopedic precast including at least a first section of interwoven strands of a thermoplastic material having a first melting temperature and at least a second section of interwoven strands of a non-thermoplastic material; and
a thermal cover configured to (i) preclude thermal energy equal to or exceeding the first melting temperature onto at least a portion of the first section of the orthopedic precast to cause the portion of the first section to remain in a first state and a remainder of the first section of the orthopedic precast, after cooling, to transition from the first state to a second state as the orthopedic precast is converted into an orthosis,
wherein the second state is more rigid than the first state.

11. The orthosis customization system of claim 10, wherein the thermal cover is made of and configured to be coupled to the orthopedic precast prior to applying the thermal energy to the orthopedic precast.

12. The orthosis customization system of claim 11, wherein the thermal cover includes an adhesive backing for coupling to a top surface of the orthopedic precast.

13. The orthosis customization system of claim 10, wherein the orthopedic precast further comprises a third section including interwoven strands of a second thermoplastic material having a second melting temperature higher than the first melting temperature so that the second portion remains in a flexible state when the orthopedic precast is converted into the orthosis.

14. The orthosis customization system of claim 10, wherein the orthopedic precast further comprises a third section including interwoven strands of the first thermoplastic material having a tighter interweaving pattern than the interwoven strands of the first thermoplastic material associated with the first section.

15. The orthosis customization system of claim 14, wherein the third section transitions to a rigid section of the orthosis having a greater rigidity than the first section in response to an application of the thermal energy being equal to or exceeding the first melting temperature onto both the first section and the third section.

16. The orthosis customization system of claim 10, wherein the orthopedic precast further comprises a third section including interwoven strands of the first thermoplastic material having a thickness in diameter greater than a thickness of the interwoven strands of the first thermoplastic material associated with the first section.

17. The orthosis customization system of claim 16, wherein the third section transitions to a rigid section of the orthosis having a greater rigidity than the first section in response to an application of the thermal energy being equal to or exceeding the first melting temperature onto both the first section and the third section.

18. The orthosis customization system of claim 10 being a lumbar sacral orthosis (LSO) or part of a thoracic lumbar sacral orthosis (TLSO).

19. A method for generating an customized orthosis, comprising:
providing an orthopedic precast that comprises a first section including interwoven strands of a first thermoplastic material having a first melting temperature;
applying a thermal cover over at least a portion of the first section;
applying thermal energy to the orthopedic precast from a thermal unit with the thermal cover positioned between the thermal unit and the first section of the orthopedic precast,
wherein the thermal cover precluding thermal energy equal to or exceeding the first melting temperature onto at least a first portion of the first section of the orthopedic precast covered by the thermal cover so that interwoven strands of the first thermoplastic material covered by the thermal cover to remain in a flexible state while interwoven strands of the first thermoplastic material associated with uncovered portion of the first section of the orthopedic precast, after cooling, transitions from the flexible state to a rigid state as the orthopedic precast is converted into an orthosis.
